(19) United States
(12) Patent Application Publication
(10) Pub. No.: US 2002/0161405 A1
(43) Pub. Date: Oct. 31, 2002

(54) Electrical youth current cure

(76) Inventor: Gene F. Drake, Lakewood, OH (US)

Correspondence Address: MR. GENE F. DRAKE, 18900 DETROIT AVENUE, EXTENSION #503, LAKEWOOD, OH, 44107, US

(21) Appl. No.: 10/096,262

(22) Filed: Mar. 12, 2002

Related U.S. Application Data
(60) Provisional application No. 60/287,470, filed on Apr. 30, 2001.

(51) Int. Cl. A61N 1/00

(52) U.S. Cl. 607/2

(57) ABSTRACT

A device for inducing an electric current in the body to reverse and cure aging and disease. The current may be induced by means of the electrochemical potential between aluminum foil tabs (10, 11 & 12) on the forehead and feet and gold and silver dental work.

### CROSS REFERENCE TO RELATED APPLICATIONS

This invention is entitled to the Provisional Patent Application Ser. No. 60/287470 filed Apr. 30, 2001.

### BACKGROUND

1. Field of Invention

This invention relates to curing diseases and reversing the aging process in animals and humans, specially to effecting these events by passing an electrical current through the body or part there of for a limited time.

2. Description of Prior Art

No art was found linking an electrical current for a limited time in the human body and reversing the aging process.

Inventors have used an electrical current for therapeutic and health promoting purposes U.S. Pat. No. 6,142,927 to Clark and Hoyt (2000) shows electrical current in digital sequences or product sequences generated by a computer to simulate a response in the human body equal to the substances being simulated. Such a device has a number of disadvantages:

(a) It requires a computer.

(b) Its use in the patent above does not produce a reversal of the aging process.

(c) It has not been shown to cure cancer, aids, arthritis, diabetes, multiple sclerosis, and heart disease, etc.

### SUMMARY

In accordance with the present invention a constant current for a limited time cures all the disadvantages above.

Objects and Advantages

In addition to the advantages shown above, the present invention offers:

(a) an impediment to all diseases;

(b) an increase in the height and size of the subject;

(c) cure in specific areas by a small electrical charge held for long periods near the affected disease areas;

(d) the use of metals in addition to gold and silver, eg. chrome to effect the electricity;

(e) the use of periodically longer periods of electrical treatment to effect greater results;

(f) the use of decreased times of electrical current as the cure progresses;

### DRAWING FIGURES

FIG. 1 is a human subject with ½″ by ½″ metal strips on his feet 11 & 12 and forehead 10 and gold and/or silver fillings in his teeth.

FIG. 2 is a metallic strip 10 with a clean side 13 and an adhesive side 14.

FIG. 3 is a view of the underside of the feet of FIG. 1 with metallic strips.

### DESCRIPTION

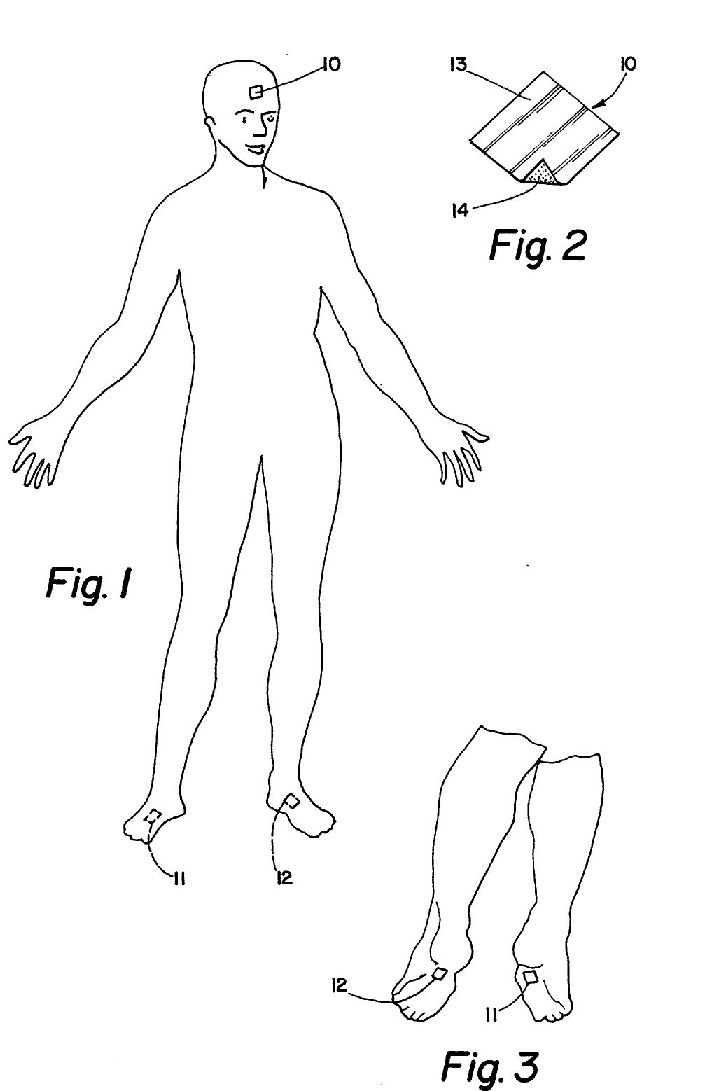

FIGS. 1, 2, & 3—Preferred Embodiment

A preferred embodiment of the invention is illustrated in FIG. 1. The teeth of the subject are filled with some gold and silver dental work. The metallic tabs 10, 11, & 12 can be aluminum foil, creating an electrochemical potential current with the gold and silver or battery contacts creating an electric current.

Advantages

From the description above, a number of advantages of the device become evident:

(a) an impediment to all diseases;

(b) cure in specific areas by placement of the tabs over diseased areas with reduced electrical charge for longer times;

(c) the use of a necklace in lieu of gold and silver in teeth of a metallic substance to generate an electrochemical potential with the tabs;

(d) the use of periodically longer periods of exposure for greater results.

(e) the use of decreased times of electrical current over the months as the cure and or reversal of aging progresses.

Operation—FIGS. 1, 2, & 3

The manner of using the device in the early months is by ¾″ by 1″ aluminum foil strips, 10, 11,& 12 to pass an electrochemical potential current through the body to gold and or silver in the teeth. The strips are applied to the body daily for 3 days, skipping the 4^{th}, for 3 or 4 hours daily. They may be applied with tape or an adhesive but serve as well under socks and a hat. The current has a fatiguing effect if used longer. After 8 months the size of the tabs is reduced to ½″ by ½″ used twice weekly. The tab on the forehead is applied for ½ hour and that to the feet for 1½ hours. Cure to a specific area can be accomplished by placement of a ¼″ by ¼″ tab over a diseased area. A gold, silver or chrome plated necklace will substitute for gold and silver dental work when that is unavailable. Periodically, biweekly for example, use of the tabs for three times the customary time is an effective booster to the action of the device. Less and less electricity will accomplish the same results as the cure and reverse of aging progresses over the months. The reduction in the size and use of the tabs in this “operation” section of the disclosure is an example.

Conclusion, Ramifications and Scope

Accordingly, the reader will see that this electrical fountain of youth is most simply used in its electrochemical potential format. As shown herein:

(a) it reverses all diseases, including age;

(b) it increases the height and size of the subject;

(c) cure at specific sites is enhanced by a small metallic tab of electrochemical potential for a more lengthy time of exposure;

(d) even a chrome plated necklace may be used when gold and silver dental work is unavailable. It must be used about twice the time used with enlarged aluminum foil tabs.

(e) periodic exposure for twice the time accelerates the cure or reverse of aging.

Although the description above contains many specifications, these should not be construed as limiting the scope of the invention but as merely providing illustrations of some of the preferred embodiments. The scope should be determined by the appended claims.

1. Any device for inducing electrical current in the body to reverse aging and disease.

2. Inducing electrical current in the body by means of an electrochemical potential such as between aluminum foil tabs 10, 11 & 12 and the gold and silver dental work.

3. Tabs of foil cut specifically to accomplish claim 2
